(19) United States
(12) Patent Application Publication
(10) Pub. No.: US 2025/0332174 A1
(43) Pub. Date: Oct. 30, 2025

(54) IVABRADINE HYDROCHLORIDE SUSTAINED-RELEASE TABLET AND PREPARATION METHOD THEREOF

(71) Applicant: JIANGSU NOVO-BITO PHAMMACEUTICAL RESEARCH CO., LTD., Wuxi (CN)

(72) Inventors: Xia LIN, Wuxi (CN); Ziyi Yang, Wuxi (CN); Chunxue Li, Wuxi (CN); Xiang Ding, Wuxi (CN); Xiaoyu Fan, Wuxi (CN); Lin Zhu, Wuxi (CN); Dongyu LI, Wuxi (CN); Junjie Zheng, Wuxi (CN); Xueqing Wu, Wuxi (CN)

(21) Appl. No.: 19/259,016

(22) Filed: Jul. 3, 2025

Related U.S. Application Data
(63) Continuation of application No. PCT/CN2024/129374, filed on Nov. 1, 2024.

(30) Foreign Application Priority Data
Sep. 8, 2023 (CN) 202311156485.9

(51) Int. Cl. A61K 31/55; A61K 9/20

(52) U.S. Cl. CPC A61K 31/55; A61K 9/2009; A61K 9/2013; A61K 9/2018; A61K 9/2054; A61K 9/2095

(57) ABSTRACT

The present disclosure discloses an Ivabradine hydrochloride sustained-release tablet and a preparation method thereof. The sustained-release tablet contains Ivabradine hydrochloride, a gel matrix sustained-release material, a pore-forming agent and a lubricating agent. The gel matrix sustained-release material is hydroxypropylmethyl cellulose. The pore-forming agent is selected from one or a mixture of microcrystalline cellulose and lactose. The lubricating agent is selected from one or a mixture of colloidal silica and magnesium stearate. The sustained-release tablet just needs to be orally administered once a day to reduce the peak-trough concentration fluctuations, thereby reducing the frequency of medication administration for patients and improve the medication compliance of the patients.

### TECHNICAL FIELD

The present disclosure belongs to the field of pharmaceutical formulations, and particularly relates to an Ivabradine hydrochloride sustained-release tablet and a preparation method thereof.

### BACKGROUND

Ischemic heart disease is still a main factor in global health loss, and the incidence of angina pectoris is increasing annually worldwide. Stable angina pectoris is the most common symptom in patients with stable coronary heart diseases. Due to recurrent myocardial ischemia, the symptoms of angina pectoris seriously restrict the daily activity level and endurance of a patient, significantly negatively impacting their quality of life.

Heart rate is a main influence factor for myocardial oxygen consumption, and increased heart rate shortens the cardiac cycle, thereby reducing systolic perfusion and then further decreasing myocardial oxygen supply. Imbalance in myocardial oxygen consumption may cause myocardial ischemia, triggering angina pectoris. Abnormal heart rate is an independent risk factor for cardiovascular system diseases. An elevated resting heart rate is positively correlated with the mortality of cardiovascular events such as a coronary heart disease, heart failure (HF) and myocardial infarction, and therefore controlling the resting heart rate has become a main targeting method for clinical treatment of cardiovascular diseases.

Ivabradine hydrochloride has a chemical name of 7,8-dimethoxyl-3-(3-[[(1S) (4,5-dimethoxylbenzocyclobutane-1-yl)methyl]-methylamino]propyl)-1,3,4,5-tetrahydro-2H-benzazepin-2-one hydrochloride, with a CAS registration number of 148849-67-6. A structural formula of Ivabradine hydrochloride is shown as follows:

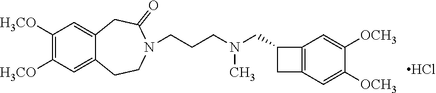 [SMILES: COc1cc2c(cc1OC)CC(=O)N(CCCN(C)C[C@H]1Cc3cc(OC)c(OC)cc31)CC2 ; Cl]

Ivabradine hydrochloride is a first funny current inhibitor with a specific heart rate-lowering effect. Ivabradine hydrochloride works by blocking IF current in a sinoatrial node, which controls the cardiac contraction and regulates the heart rate. When the IF current is blocked, heart rate decreases, reducing stress on the heart. Clinically, Ivabradine hydrochloride is mainly used for patients with chronic heart failures (NYHA class II-IV) who have sinus rhythm and the heart rate of ≥75 beats/min, accompanied by systolic dysfunction, or is used for contraindications or intolerance to beta-receptor blocker treatment if being combined with a standard therapy (including a beta-receptor blocker). At present, Ivabradine hydrochloride on the market is in a dosage form of a tablet with specifications of 5 mg and 7.5 mg, and taken orally twice a day with meals in the morning and evening. The currently listed Ivabradine hydrochloride is available in the United States, Japan, Europe and other countries.

Almost 45,000 cases of patients have participated in clinical study on Ivabradine hydrochloride tablets. The most common adverse reactions are a flicker phenomenon (phosphenes) and bradycardia, which are dose-dependent. Among them, 3.3% of patients reported bradycardia, especially within initial 2-3 months after treatment starts, while 0.5% of patients experienced serious bradycardia (≤40 beats/min). This is mainly because after the Ivabradine hydrochloride tablet is orally administered, a plasma concentration rapidly reaches a peak, which leads to a rapid decrease in heart rate and great fluctuations in plasma concentration. Therefore, developing a once-daily sustained-release formulation of ivabradine hydrochloride is of great significance in reducing trough plasma concentration fluctuations, minimizing adverse effects, and improving medication compliance.

Patent application CN1482901A discloses a solid pharmaceutical composition for controlling the release of Ivabradine hydrochloride. In this application, a mixture of two polymethacrylates (Eudragit® RL and RS) and ivabradine hydrochloride is heated to 80-130° C. to prepare a sustained-release mixture, and the releasing rate of the drug is adjusted by changing the proportions of the two polymers. This method involves a complex preparation process and is not conducive to industrial-scale production.

Patent application CN102908327A discloses Ivabradine hydrochloride and a sustained-release formulation of a medicinal salt thereof. A sustained-release matrix material is selected from one or a mixture of polyoxyethylene, polyvinyl acetate and polyvinylpyrrolidone polymers. The sustained-release tablet prepared by using hydroxypropylmethyl cellulose K100M can only achieve an 8-hour sustained release. The sustained-release effect is achieved by using high-molecular-weight polyoxyethylene (a molecular weight of greater than 1,000,000 Da) as a sustained-release material, Ivabradine hydrochloride as an active ingredient and meanwhile adding another water-insoluble wax matrix material glyceryl behenate. Furthermore, compared with an immediate-release tablet, the peak concentration of the above-mentioned sustained-release tablet is reduced by about 75% after being orally administrated to a Beagle dog, which easily leads to too low peak concentration and poor curative effect; in addition, the present disclosure alters the salt form of Ivabradine, and its impact on the in-vivo pharmacological activity of the compound cannot be predicted.

### SUMMARY

The objective of this section is to summarize some aspects of embodiments of the present disclosure and briefly introduce some preferred embodiments. Simplification or omission may be made to the abstract of the specification and invention title to avoid blurring the objective of this section, the abstract of the specification and invention title, and such the simplification or omission cannot be used for limiting the scope of the present disclosure.

In view of the problems as described above and/or in the prior art, the present disclosure is proposed.

Therefore, the objective of the present disclosure is to overcome the defects in the prior art to provide an Ivabradine hydrochloride sustained-release tablet, comprising:
Ivabradine hydrochloride, a gel matrix sustained-release material, a pore-forming agent and a lubricating agent;
wherein, the mass parts of raw materials are as follows:
5-15 parts of Ivabradine hydrochloride;
80-140 parts of gel matrix sustained-release material;
46-112 parts of pore-forming agent; and
4-12 parts of lubricating agent;
the gel matrix sustained-release material is hydroxypropylmethyl cellulose which is selected from a mixture of two hydroxypropylmethyl celluloses with viscosity indication values of 100000 mPa·s and 5 mPa·s respectively, and a weight ratio of hydroxypropylmethyl cellulose with the viscosity indication value of 100000 mPa·s and hydroxypropylmethyl cellulose with the viscosity indication value of 5 mPa·s is 3:1-1:4.

As a preferred embodiment of the Ivabradine hydrochloride sustained-release tablet of the present disclosure, the weight ratio of the hydroxypropylmethyl cellulose with the viscosity indication value of 100000 mPa·s and the hydroxypropylmethyl cellulose with the viscosity indication value of 5 mPa·s is 1:2-1:3.

As a preferred embodiment of the Ivabradine hydrochloride sustained-release tablet of the present disclosure, a weight percentage of the gel matrix sustained-release material in the sustained-release tablet is 40%-70%.

As a preferred embodiment of the Ivabradine hydrochloride sustained-release tablet of the present disclosure, the weight percentage of the gel matrix sustained-release material in the sustained-release tablet is 50%-60%.

As a preferred embodiment of the Ivabradine hydrochloride sustained-release tablet of the present disclosure, the pore-forming agent is selected from one or a mixture of microscystallone and lactose.

As a preferred embodiment of the Ivabradine hydrochloride sustained-release tablet of the present disclosure, the pore-forming agent is a mixture of microscystallone and lactose, wherein a weight percentage of the microscystallone in the sustained-release tablet is 0%-33%, and a weight percentage of the lactose in the sustained-release tablet is 0%-33%.

As a preferred embodiment of the Ivabradine hydrochloride sustained-release tablet of the present disclosure, the weight percentage of the microscystallone in the sustained-release tablet is 8%-25%, and the weight percentage of the lactose in the sustained-release tablet is 8%-25%.

As a preferred embodiment of the Ivabradine hydrochloride sustained-release tablet of the present disclosure, the lubricating agent is selected from one or a mixture of colloidal silica and magnesium stearate.

As a preferred embodiment of the Ivabradine hydrochloride sustained-release tablet of the present disclosure, a preparation method of an Ivabradine hydrochloride sustained-release tablet comprises: initially mixing Ivabradine hydrochloride with a pore-forming agent and then mixing the above-mentioned mixture with a gel matrix material; and then adding a lubricating agent for final mixing, and performing tableting to obtain the Ivabradine hydrochloride sustained-release tablet.

### The Present Disclosure has the Beneficial Effects

By screening a series of sustained-release matrix materials, it is found that the Ivabradine hydrochloride sustained-release tablet that tableting using a mixture of hydroxypropylmethyl celluloses with different molecular weights as the sustained-release matrix material and a mixture of microcrystalline cellulose and lactose as the pore-forming agent, a 24-hour sustained-release ivabradine hydrochloride tablet can be prepared simply by conventional direct powder compression method, with a simple process suitable industrial production; the prepared Ivabradine hydrochloride sustained-release tablet has an obvious in-vivo sustained release effect and needs to be orally administered once a day without administration many times; the peak concentration is reduced to about 50% that of the immediate-release tablet, which still maintain its in-vivo pharmacological effect; and the salt form of Ivabradine is not altered.

### BRIEF DESCRIPTION OF THE DRAWINGS

To more clearly illustrate the technical solution in embodiments of the present disclosure, drawings required to be used in embodiments will be simply introduced, obviously, the drawings in the following description are merely some embodiments of the present disclosure, and persons of ordinary skill in the art can also obtain other drawings according to these drawings without creative efforts.

FIG. 1 is a comparison graph of dissolution curves in Comparative examples 1 and 2, and Examples 15, 21, 30 and 33;

FIG. 2 is an average plasma concentration-time curve graph of Ivabradine hydrochloride after 5 mg of Example 30 and an Ivabradine hydrochloride tablet (2.5 mg/time, BID) were orally administrated to a Beagle dog;

FIG. 3 is an average plasma concentration-time curve graph of Ivabradine hydrochloride after 10 mg of Example 21 and an Ivabradine hydrochloride tablet (5 mg/time, BID) were orally administrated to a Beagle dog;

FIG. 4 is an average plasma concentration-time curve graph of Ivabradine hydrochloride after 15 mg of Example 33 and an Ivabradine hydrochloride tablet (7.5 mg/time, BID) were orally administrated to a Beagle dog; and

FIG. 5 is an average plasma concentration-time curve graph of an Ivabradine hydrochloride sustained-release formulation in patent application CN102908327A.

### DETAILED DESCRIPTION OF THE EMBODIMENTS

To make the above purpose, features and advantages of the present disclosure more obvious and understandable, specific embodiments of the present disclosure will be described in detail in combination with embodiments of the specification.

The following description states many specific details so as to sufficiently understand the present disclosure, but the present disclosure can also be implemented by adopting other embodiments different from those in this description, those skilled in the art make similar promotion without obeying the connotation of the present disclosure, and therefore the present disclosure is not limited by specific embodiments disclosed hereinafter.

Further, a phrase “one embodiment” or “embodiment” here refers to specific feature, structure and characteristic contained in at least one implementation mode of the present disclosure. The phrase “in one embodiment” occurring in different places of this specification does not refer to the same embodiment, nor is it a separate or selective embodiment that is mutually exclusive with other embodiments.

### Examples 1-6

### Comparison of Sustained-Release Material Types:

Sustained-release tablets were prepared according to ingredients and amounts shown in Table 1. The amount shown in Table 1 represents a weight (mg) of each ingredient in each tablet. For each example, raw materials and excipients were evenly mixed and then compressed into a tablet with a weight of about 200 mg by using a tableting press. The formulation is seen in Table 1.

TABLE 1

| | Mass of each ingredient (mg/tablet) | | | | | |
|---|---|---|---|---|---|---|
| | Example | Example | Example | Example | Example | Example |
| Ingredients | 1 | 2 | 3 | 4 | 5 | 6 |
| Ivabradine 1 | 10 | 10 | 10 | 10 | 10 | 10 |
| Polyoxyethylene | 176 | — | — | — | — | — |
| Kollidon SR | — | 176 | — | — | — | — |
| Glyceryl behenate | 10 | 10 | — | — | — | 186 |
| Hydroxypropylmethyl | — | — | 186 | — | 176 | — |
| cellulose HPMC | | | | | | |
| K100M | | | | | | |
| Hydroxypropylmethyl | — | — | — | 186 | 10 | — |
| cellulose HPMC | | | | | | |
| E5 | | | | | | |
| Colloidal silica | 2 | 2 | 2 | 2 | 2 | 2 |
| Magnesium stearate | 2 | 2 | 2 | 2 | 2 | 2 |
| Total weight | 200 | 200 | 200 | 200 | 200 | 200 |

Note 1
The active ingredient was fed in a form of hydrochloride.

A dissolution experiment was performed on the tablets prepared in Examples 1-6 in accordance with Apparatus 1 (Basket Apparatus) of General Rule 0931 “Dissolution and Drug Release Test” from Volume IV of the Chinese Pharmacopoeia 2020 Edition. The dissolution experiment was performed by using 900 ml of purified water as a dissolution medium at 37±0.5° C. at the rotational speed of 75 rpm. Small portions of samples were respectively taken from the dissolution medium at 1, 2, 4, 6, 8, 12 and 24 hours. Each portion of sample was detected using a high-performance liquid chromatograph (HPLC, a detection wavelength: 220 nm) to calculate the releasing rate. The results are shown in Table 2.

TABLE 2

| | Releasing rate (%) | | | | | |
|---|---|---|---|---|---|---|
| Time | Example | Example | Example | Example | Example | Example |
| (h) | 1 | 2 | 3 | 4 | 5 | 6 |
| 1 | 12.8 | 12.5 | 9.6 | 45.7 | 14.3 | 10.5 |
| 2 | 18.5 | 18.0 | 14.1 | 81.7 | 23.1 | 13.1 |
| 4 | 26.1 | 24.5 | 17.6 | 98.4 | 35.5 | 15.6 |
| 6 | 33.6 | 29.6 | 22.1 | 99.5 | 46.0 | 18.2 |
| 8 | 39.8 | 33.9 | 26.5 | — | 54.3 | 22.5 |
| 12 | 56.7 | 39.4 | 30.3 | — | 65.2 | 25.3 |
| 16 | 77.3 | 45.4 | 33.5 | — | 76.1 | 26.9 |
| 24 | 84.6 | 53.6 | 37.7 | — | 88.4 | 28.3 |

It can be seen from Table 2 that samples using polyoxyethylene, Kollidon SR and behenate glycerol ester as the sustained-release matrices (Examples 1, 2 and 6) have relatively slow dissolution rates and cannot be completely dissolved out within 24 hours; after a tablet solely using HPMC K100M as a sustained-release material (Example 3) is dissolved out and released for 24 hours, there are still drug residues inside the core so as to form incomplete release, thereby creating an unsatisfactory sustained-release effect; and after a tablet solely using HPMC E5 as a sustained-release material (Example 4) is dissolved out and released for 4 hours, there are no flaky substances in a dissolution cup, so as not to take an obvious sustained-release effect. The mixture of HPMC K100M and HPMC E5 is used as the sustained-release matrix material (Example 5) is adopted, and a sustained-release formulation having a good sustained release effect can be prepared by using a powder direct compression process. Furthermore, compared with a formulation particle in patent application CN102908327A, this formulation particle has better flowability and compressibility, and no sticky phenomenon appears during the tableting.

### Examples 7-11

### Comparison of Types and Amounts of Pore-Forming Agents:

Sustained-release tablets were prepared according to ingredients and amounts shown in Table 3. The amount shown in Table 3 represents a weight (mg) of each ingredient in each tablet. For each example, raw materials and excipients were evenly mixed and then compressed into a tablet with a weight of about 200 mg by using a tableting press. The formulation is seen in Table 3.

TABLE 3

| | Mass of each ingredient (mg/tablet) | | | | |
|---|---|---|---|---|---|
| | Example | Example | Example | Example | Example |
| Ingredients | 7 | 8 | 9 | 10 | 11 |
| Ivabradine 1 | 10 | 10 | 10 | 10 | 10 |
| Hydroxypropylmethyl | 60 | 60 | 60 | 60 | 60 |
| cellulose HPMC | | | | | |
| K100M | | | | | |
| Hydroxypropylmethyl | 60 | 60 | 60 | 60 | 60 |
| cellulose HPMC E5 | | | | | |
| Microcrystalline | 66 | — | 16 | 33 | 50 |
| cellulose | | | | | |
| Lactose | — | 66 | 50 | 33 | 16 |
| Colloidal silica | 2 | 2 | 2 | 2 | 2 |
| Magnesium stearate | 2 | 2 | 2 | 2 | 2 |
| Total weight | 200 | 200 | 200 | 200 | 200 |

Note 1:
The active ingredient was fed in a form of hydrochloride.

A dissolution experiment was performed on the tablets prepared in Examples 7-11 in accordance with Apparatus 1 (Basket Apparatus) of General Rule 0931 “Dissolution and Drug Release Test” from Volume IV of the Chinese Pharmacopoeia 2020 Edition. The dissolution experiment was performed by using 900 ml of purified water as a dissolution medium at 37±0.5° C. at the rotation speed of 75 rpm. Small portions of samples were respectively taken from the dissolution medium at 1, 2, 4, 6, 8, 12 and 24 hours. Each portion of sample was detected using a high-performance liquid chromatograph (HPLC, a detection wavelength: 220 nm) to calculate a releasing rate. The results are shown in Table 4.

TABLE 4

| | Releasing rate (%) | | | | |
|---|---|---|---|---|---|
| | Example | Example | Example | Example | Example |
| Time (h) | 7 | 8 | 9 | 10 | 11 |
| 1 | 17.4 | 22.3 | 21.2 | 21.1 | 19.1 |
| 2 | 26.4 | 32.6 | 30.9 | 30.7 | 29.8 |
| 4 | 43.5 | 50.2 | 48.8 | 49.6 | 45.3 |
| 6 | 55.2 | 62.3 | 61.3 | 63.5 | 57.0 |
| 8 | 65.3 | 77.8 | 75.8 | 75.7 | 66.1 |
| 12 | 80.5 | 91.4 | 86.5 | 89.2 | 79.1 |
| 16 | 90.1 | 95.2 | 94.7 | 95.8 | 87.0 |
| 24 | 95.6 | 97.9 | 98.6 | 96.6 | 94.5 |

It can be seen from Table 4 that a sustained-release formulation having a good sustained-release effect is prepared by using a powder direct compression process with a mixture of microcrystalline cellulose and lactose in a ratio of 1:1 as a pore-forming agent (Example 10). Both the flowability and compressibility of the formulation particle are good.

### Examples 12-22

### Comparison of Amounts and Proportions of Sustained-Release Materials

Sustained-release tablets were prepared according to ingredients and amounts shown in Table 5-Table 6. The amount in Table 5-Table 6 represents a weight (mg) of each ingredient in every tablet. For each example, raw materials and excipients were evenly mixed and then compressed into a tablet with a weight of about 200 mg by using a tableting press. The formulation is seen in Tables 5-6.

TABLE 5

| | Mass of each ingredient (mg/tablet) | | | | | |
|---|---|---|---|---|---|---|
| | Example | Example | Example | Example | Example | Example |
| Ingredients | 12 | 13 | 14 | 15 | 16 | 17 |
| Ivabradine 1 | 10 | 10 | 10 | 10 | 10 | 10 |
| Hydroxypropylmethyl | 40 | 20 | 50 | 34 | 25 | 20 |
| cellulose HPMC | | | | | | |
| K100M | | | | | | |
| Hydroxypropylmethyl | 40 | 60 | 50 | 66 | 75 | 80 |
| cellulose HPMC E5 | | | | | | |
| Microcrystallone | 53 | 53 | 43 | 43 | 43 | 43 |
| cellulose | | | | | | |
| Lactose | 53 | 53 | 43 | 43 | 43 | 43 |
| Colloidal silica | 2 | 2 | 2 | 2 | 2 | 2 |
| Magnesium stearate | 2 | 2 | 2 | 2 | 2 | 2 |
| Total weight | 200 | 200 | 200 | 200 | 200 | 200 |
| Total amount % of | 40 | 40 | 50 | 50 | 50 | 50 |
| gel matrix material | | | | | | |
| K100M:E5 | 1:1 | 1:3 | 1:1 | 1:2 | 1:3 | 1:4 |

Note 1:
The active ingredient was fed in a form of hydrochloride.

TABLE 6

| | Mass of each ingredient (mg/tablet) | | | | |
|---|---|---|---|---|---|
| | Example | Example | Example | Example | Example |
| Ingredients | 18 | 19 | 20 | 21 | 22 |
| Ivabradine 1 | 10 | 10 | 10 | 10 | 10 |
| Hydroxypropylmethyl | 90 | 80 | 40 | 30 | 70 |
| cellulose HPMC | | | | | |
| K100M | | | | | |
| Hydroxypropylmethyl | 30 | 40 | 80 | 90 | 70 |
| cellulose HPMC E5 | | | | | |
| Microcrystallone | 33 | 33 | 33 | 33 | 23 |
| cellulose | | | | | |
| Lactose | 33 | 33 | 33 | 33 | 23 |
| Colloidal silica | 2 | 2 | 2 | 2 | 2 |
| Magnesium stearate | 2 | 2 | 2 | 2 | 2 |
| Total weight | 200 | 200 | 200 | 200 | 200 |
| Total amount % of | 60 | 60 | 60 | 60 | 70 |
| hydroxypropylmethyl | | | | | |
| cellulose | | | | | |
| K100M: E5 | 3:1 | 2:1 | 1:2 | 1:3 | 1:1 |

Note 1:
The active ingredient was fed in a form of hydrochloride.

A dissolution experiment was performed on the tablets prepared in Examples 12-22 in accordance with Apparatus 1 (Basket Apparatus) of General Rule 0931 “Dissolution and Drug Release Test” from Volume IV of the Chinese Pharmacopoeia 2020 Edition. The dissolution experiment was performed by using 900 ml of purified water as a dissolution medium at 37±0.5° C. at the rotation speed of 75 rpm. Small portions of samples were respectively taken from the dissolution medium at 1, 2, 4, 6, 8, 12 and 24 hours. Each portion of sample was detected using a high-performance liquid chromatograph (HPLC, a detection wavelength: 220 nm) to calculate a releasing rate. The results are shown in Table 7.

TABLE 7

| | Releasing rate (%) | | | | | | | | | | |
|---|---|---|---|---|---|---|---|---|---|---|---|
| | Example | Example | Example | Example | Example | Example | Example | Example | Example | Example | Example |
| Time (h) | 12 | 13 | 14 | 15 | 16 | 17 | 18 | 19 | 20 | 21 | 22 |
| 1 | 25.4 | 30.5 | 23.6 | 25.1 | 27.2 | 28.9 | 20.8 | 23.9 | 24.9 | 24.5 | 22.4 |
| 2 | 38.6 | 45.7 | 36.6 | 40.5 | 41.7 | 44.3 | 33.1 | 35.1 | 37.0 | 38.8 | 35.5 |
| 4 | 57.3 | 66.1 | 55.0 | 59.4 | 62.4 | 65.5 | 50.4 | 53.4 | 55.4 | 55.2 | 53.9 |
| 6 | 70.6 | 81.6 | 67.7 | 72.5 | 78.0 | 80.1 | 65.5 | 66.5 | 69.7 | 72.6 | 68.2 |
| 8 | 82.5 | 90.0 | 79.3 | 85.8 | 88.1 | 89.6 | 74.1 | 72.3 | 81.7 | 82.1 | 78.1 |
| 12 | 90.7 | 95.4 | 88.6 | 92.4 | 94.9 | 95.6 | 83.8 | 84.3 | 91.4 | 91.1 | 89.0 |
| 16 | 96.9 | 97.6 | 94.5 | 97.6 | 96.8 | 97.0 | 92.4 | 92.0 | 95.3 | 95.4 | 93.1 |
| 24 | 97.2 | 97.9 | 96.5 | 98.2 | 96.8 | 97.2 | 95.9 | 97.7 | 95.9 | 96.3 | 97.4 |

It can be seen from Table 7 that the total amount percentage of the hydroxypropylmethyl cellulose is between 50% and 60%, and furthermore when the ratio of HPMC K100M: HPMC E5 is 1:2 to 1:3 (Examples 15, 16, 20 and 21), Ivabradine hydrochloride formulations having a good sustained release effect are prepared by using a powder direct compression process and have in-vitro release characteristics of less than 35% release amount of an active ingredient at 1 hour (i.e., Ivabradine), 50%-80% release amount (accumulated releasing rate) of the active ingredient at 4-6 hours and over 85% release amount (accumulated releasing rate) of the active ingredient at 12 hours.

### Examples 23-27

### Comparison of Amounts and Proportions of Lubricating Agents

Sustained-release tablets were prepared according to ingredients and amounts shown in Table 8. The amount shown in Table 8 represents a weight (mg) of each ingredient in each tablet. For each example, raw materials and excipients were evenly mixed and then compressed into a tablet with a weight of about 200 mg by using a tableting press. The formulation is seen in Table 8.

TABLE 8

| | Mass of each ingredient (mg/tablet) | | | | | |
|---|---|---|---|---|---|---|
| | Example | Example | Example | Example | Example | Example |
| Ingredients | 21 | 23 | 24 | 25 | 26 | 27 |
| Ivabradine 1 | 10 | 10 | 10 | 10 | 10 | 10 |
| Hydroxypropylmethyl | 30 | 30 | 30 | 30 | 30 | 30 |
| cellulose HPMC | | | | | | |
| K100M | | | | | | |
| Hydroxypropylmethyl | 90 | 90 | 90 | 90 | 90 | 90 |
| cellulose HPMC E5 | | | | | | |
| Microcrystalline | 33 | 31 | 31 | 29 | 29 | 25 |
| cellulose | | | | | | |
| Lactose | 33 | 33 | 33 | 33 | 33 | 33 |
| Colloidal silica | 2 | 3 | 5 | 4 | 6 | 6 |
| Magnesium stearate | 2 | 3 | 1 | 4 | 2 | 6 |
| Total weight | 200 | 200 | 200 | 200 | 200 | 200 |
| A proportion % of a | 2 | 3 | 3 | 4 | 4 | 6 |
| lubricating agent in | | | | | | |
| the formulation | | | | | | |

Note 1:
The active ingredient was fed in a form of hydrochloride.

The tablet extrusion forces and hardness of samples in Examples 21, 23 and 27 during the tableting were monitored in real time. The monitoring results are seen in Table 9.

TABLE 9

| | Example | Example | Example | Example | Example | Example |
|---|---|---|---|---|---|---|
| Monitoring item | 21 | 23 | 24 | 25 | 26 | 27 |
| Average tablet | 6 | 6 | 7 | 5 | 6 | 5 |
| extrusion force (N) | | | | | | |
| Average hardness | 7.94 | 8.02 | 7.56 | 6.89 | 6.52 | 6.34 |
| (n = 10) (kg) | | | | | | |

For samples in Examples 21 and 23 to 27, 10 tablets were taken and their contents were respectively detected. The detection results are seen in Table 10.

TABLE 10

| | Example | Example | Example | Example | Example | Example |
|---|---|---|---|---|---|---|
| Name | 21 | 23 | 24 | 25 | 26 | 27 |
| Average | 99.6 | 99.2 | 98.9 | 99.5 | 98.7 | 98.6 |
| content (n = 10) | | | | | | |
| (%) | | | | | | |
| RSD (%) | 0.83 | 0.65 | 0.88 | 0.75 | 0.82 | 0.53 |

The micromeritic characteristics of formulation particles in Examples 21 and 23 to 27 were detected by using an intelligent micromeritic tester. The detection results are seen in Table 1.

TABLE 11

| | Example | Example | Example | Example | Example | Example |
|---|---|---|---|---|---|---|
| Items | 21 | 23 | 24 | 25 | 26 | 27 |
| Angle of repose | 40.53° | 39.32° | 38.48° | 38.11° | 36.43° | 36.55° |
| Apparent density | 0.45 | 0.44 | 0.42 | 0.42 | 0.39 | 0.38 |
| (g/cm³) | | | | | | |
| Tap density | 0.65 | 0.63 | 0.63 | 0.61 | 0.59 | 0.59 |
| (g/cm³) | | | | | | |
| Compressibility | 31 | 30 | 33 | 31 | 34 | 36 |
| (%) | | | | | | |

It can be seen from the detection results in Table 9-Table 11 that when the total amount of the lubricating agent is between 2% and 3% (Examples 21, 23 and 24), the formulation particles have good flowability and compressibility, so that the Ivabradine sustained-release formulation meets the requirements on the preparation process of the present disclosure.

### Examples 28-33

### Comparison of Amounts of Active Substance:

Sustained-release tablets were prepared according to ingredients and amounts shown in Table 12. The amount shown in Table 12 represents a weight (mg) of each ingredient in each tablet. For each example, raw materials and excipients were evenly mixed and then compressed into a tablet with a weight of about 200 mg by using a tableting press. The formulation is seen in Table 12.

TABLE 12

| | Mass of each ingredient (mg/tablet) | | | | | |
|---|---|---|---|---|---|---|
| | Example | Example | Example | Example | Example | Example |
| Ingredients | 28 | 29 | 30 | 31 | 32 | 33 |
| Ivabradine 1 | 5 | 5 | 5 | 15 | 15 | 15 |
| Hydroxypropylmethyl | 20 | 25 | 30 | 20 | 25 | 30 |
| cellulose HPMC | | | | | | |
| K100M | | | | | | |
| Hydroxypropylmethyl | 60 | 75 | 90 | 60 | 75 | 90 |
| cellulose HPMC E5 | | | | | | |
| Microcrystalline | 58 | 48 | 38 | 48 | 38 | 28 |
| cellulose | | | | | | |
| Lactose | 53 | 43 | 33 | 53 | 43 | 33 |
| Colloidal silica | 2 | 2 | 2 | 2 | 2 | 2 |
| Magnesium stearate | 2 | 2 | 2 | 2 | 2 | 2 |
| Total weight | 200 | 200 | 200 | 200 | 200 | 200 |

Note 1
The active ingredient was fed in a form of hydrochloride.

A dissolution experiment was performed on the tablets prepared in Examples 28-33 in accordance with Apparatus 1 (Basket Apparatus) of General Rule 0931 “Dissolution and Drug Release Test” from Volume IV of the Chinese Pharmacopoeia 2020 Edition. The dissolution experiment was performed by using 900 ml of purified water as a dissolution medium at 37±0.5° C. at the rotation speed of 75 rpm. Small portions of samples were respectively taken from the dissolution medium at 1, 2, 4, 6, 8, 12 and 24 hours. Each portion of sample was detected using a high-performance liquid chromatograph (HPLC, a detection wavelength: 220 nm) to calculate a releasing rate. The results are seen in Table 13.

TABLE 13

| | Releasing rate (%) | | | | | |
|---|---|---|---|---|---|---|
| Time | Example | Example | Example | Example | Example | Example |
| (h) | 28 | 29 | 30 | 31 | 32 | 33 |
| 1 | 26.3 | 23.1 | 21.3 | 33.6 | 29.2 | 26.1 |
| 2 | 39.5 | 35.4 | 31.7 | 47.0 | 44.7 | 42.6 |
| 4 | 60.5 | 55.4 | 50.2 | 65.7 | 62.6 | 61.4 |
| 6 | 75.4 | 71.2 | 65.5 | 80.0 | 77.9 | 76.0 |
| 8 | 85.9 | 82.4 | 76.8 | 91.2 | 88.5 | 87.1 |
| 12 | 94.6 | 93.5 | 89.3 | 96.6 | 95.9 | 96.7 |
| 16 | 96.1 | 97.3 | 95.5 | 98.3 | 97.8 | 97.1 |
| 24 | 96.5 | 98.7 | 96.2 | 98.5 | 98.9 | 99.0 |

It can be seen from Table 13 that the Ivabradine formulation having a good sustained-release effect is prepared by using a powder direct compression process and 5 mg and 15 mg of active substances based on the mixture of HPMC K100M and HPMC E5 in the ratio of 1:3 as the sustained-release matrix material.

### Comparative Examples 1-2

Ivabradine hydrochloride sustained-release tablets were prepared by using a powder direct press method in patent application CN102908327A. The formulation is seen in Table 14.

TABLE 14

Mass of each ingredient (mg/tablet)

| | Comparative | Comparative |
|---|---|---|
| Ingredients | Example 1 | example 2 |
| Ivabradine 1 | 15 | 15 |
| Polyox303 (a molecular | 180 | — |
| weight of 7000000Da) | | |
| A mixture of polyvinyl | — | 180 |
| acetate and PVP (Kollidon | | |
| SR) | | |
| Glyceryl behenate | 20 | 20 |
| Magnesium stearate | 2 | 2 |
| Total weight | 217 | 217 |

Note 1:
The active ingredient was fed in a form of hydrochloride.

### Preparation Method:

Raw materials and excipients were evenly mixed and then directly tableted to prepare a sustained-release tablet. Through the recurrence of the preparation method in this patent, raw materials and excipients were simply mixed, and the flowability of the particle was poor. Due to excessive fine powders and the limited variety of excipients, the tablet hardness was low and the compressibility was poor during the tableting, accompanied by sticking phenomenon. The releasing rate of the prepared sustained-release tablet in 900 ml of water medium was determined by using a high-performance liquid phase chromatograph (HPLC, detection wavelength: 220 nm) assay. The result is seen in Table 15.

TABLE 15

Releasing rate (%)

| Time (h) | Comparative example 1 | Comparative example 2 |
|---|---|---|
| 1 | 11.6 | 13.3 |
| 2 | 22.1 | 27.7 |
| 4 | 37.5 | 44.5 |
| 6 | 49.3 | 60.1 |
| 8 | 62.2 | 71.3 |
| 12 | 80.4 | 84.2 |
| 16 | 91.2 | 96.2 |
| 24 | 96.5 | 97.4 |

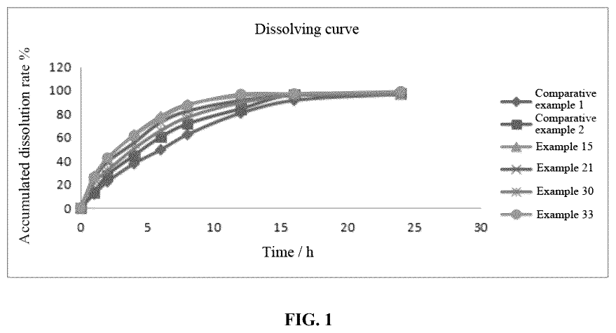

It can be seen from Table 15 that the Ivabradine hydrochloride sustained-release tablet prepared by using the method described in patent application CN102908327A has a sustained-release effect, and can achieve 16 to 24-hour sustained release. The comparison of dissolution curves in Comparative examples 1 and 2 and Examples 15. 21, 30 and 33 is seen in FIG. 1.

It can be seen from FIG. 1 that the early-stage dissolution rates in Examples 15, 21, 30 and 33 are quicker than those in Comparative examples 1 and 2, indicating that the drug can be released in a body more rapidly, thereby quickly achieving an effective treatment concentration.

### Powder Characterization

The micromeritic characteristics of the formulation particles in Comparative examples 1 and 2 and Examples 10, 13, 16 and 21 were detected by using an intelligent powder tester. The detection result is seen in Table 16.

TABLE 16

| | Comparative | Comparative | | | | |
|---|---|---|---|---|---|---|
| | example | example | Example | Example | Example | Example |
| Items | 1 | 2 | 10 | 13 | 16 | 21 |
| Angle of repose | 44.14° | 43.29° | 38.64° | 37.16° | 38.97° | 40.53° |
| Apparent | 0.41 | 0.34 | 0.44 | 0.41 | 0.43 | 0.45 |
| density (g/cm³) | | | | | | |
| Tap density | 0.66 | 0.52 | 0.63 | 0.56 | 0.61 | 0.65 |
| (g/cm³) | | | | | | |
| Compressibility | 38 | 36 | 30 | 27 | 30 | 31 |
| (%) | | | | | | |

It can be seen from Table 16 that the particle flowability in Examples 10, 13, 16 and 21 is superior to that of sample particles in Comparative examples 1 and 2.

### Stability Test:

After being packaged using aluminum bags, products in Comparative examples 1 and 2 and Examples 16, 21, 30 and 33 were placed for 1 month, 3 months and 6 months under the condition of 40° C./RH75%, and for 1 month, 3 months, 6 months and 12 months under the condition of 25° C./RH60%, and then stability investigation was performed on the above-mentioned samples. The investigation results are seen in Table 17-Table 18.

TABLE 17

| | | Relevant substances (%) | | | | | |
|---|---|---|---|---|---|---|---|
| | | Comparative | | Comparative | | | |
| | | Example 1 | | example 2 | | Example 16 | |
| | | Maximum | | Maximum | | Maximum | |
| | | single | Total | single | Total | single | Total |
| Conditions | Time | impurity | impurity | impurity | impurity | impurity | impurity |
| 0 day | | 0.07 | 0.21 | 0.09 | 0.26 | 0.04 | 0.13 |
| 40° C. | 1 | 0.16 | 0.38 | 0.18 | 0.41 | 0.02 | 0.11 |
| RH75% | months | | | | | | |
| | 3 | 0.46 | 0.75 | 0.39 | 0.68 | 0.07 | 0.19 |
| | months | | | | | | |
| | 6 | 0.66 | 1.03 | 0.71 | 1.12 | 0.08 | 0.34 |
| | months | | | | | | |
| 25° C. | 3 | 0.15 | 0.36 | 0.21 | 0.45 | 0.06 | 0.11 |
| RH60% | months | | | | | | |
| | 6 | 0.18 | 0.51 | 0.23 | 0.49 | 0.03 | 0.10 |
| | months | | | | | | |
| | 12 | 0.19 | 0.56 | 0.23 | 0.53 | 0.05 | 0.15 |
| | months | | | | | | |

TABLE 18

| | | Relevant substances (%) | | | | | |
|---|---|---|---|---|---|---|---|
| | | Example 21 | | Example 30 | | Example 33 | |
| | | Maximum | | Maximum | | Maximum | |
| | | single | Total | single | Total | single | Total |
| Conditions | Time | impurity | impurity | impurity | impurity | impurity | impurity |
| 0 day | | 0.03 | 0.11 | 0.02 | 0.10 | 0.05 | 0.14 |
| 40° C. | 1 | 0.05 | 0.12 | 0.06 | 0.15 | 0.05 | 0.17 |
| RH75% | month | | | | | | |
| | 3 | 0.07 | 0.18 | 0.07 | 0.35 | 0.08 | 0.24 |
| | months | | | | | | |
| | 6 | 0.09 | 0.36 | 0.13 | 0.48 | 0.09 | 0.40 |
| | months | | | | | | |
| 25° C. | 3 | 0.05 | 0.12 | 0.06 | 0.26 | 0.06 | 0.14 |
| RH60% | months | | | | | | |
| | 6 | 0.04 | 0.11 | 0.04 | 0.14 | 0.08 | 0.17 |
| | months | | | | | | |
| | 12 | 0.06 | 0.17 | 0.06 | 0.24 | 0.07 | 0.19 |
| | months | | | | | | |

It can be seen from Tale 17-Table 18 that samples (Comparative examples 1 and 2) prepared by using the formulations in patent application CN102908327A show a significant increase in relevant substances during the stability experiment, while the sustained-release formulation of the present disclosure only shows a slight increase in relevant substances under the condition of 40° C./RH75% and almost no increase under the condition of 25° C./RH60%, and its stability is significantly better than that of the formulation in patent application CN102908327A.

### Pharmacokinetic Study:

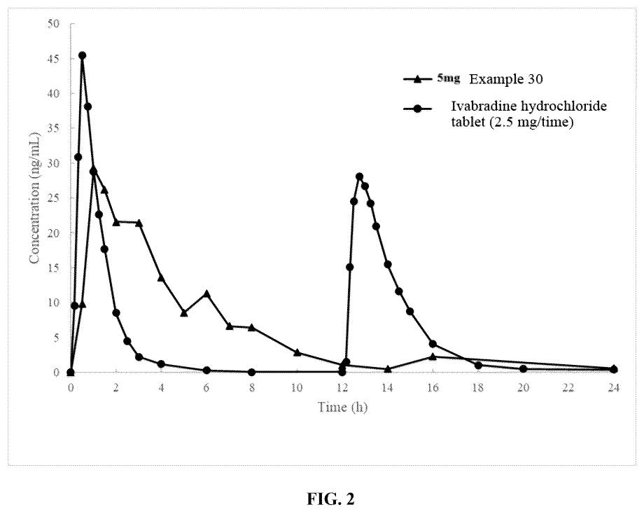

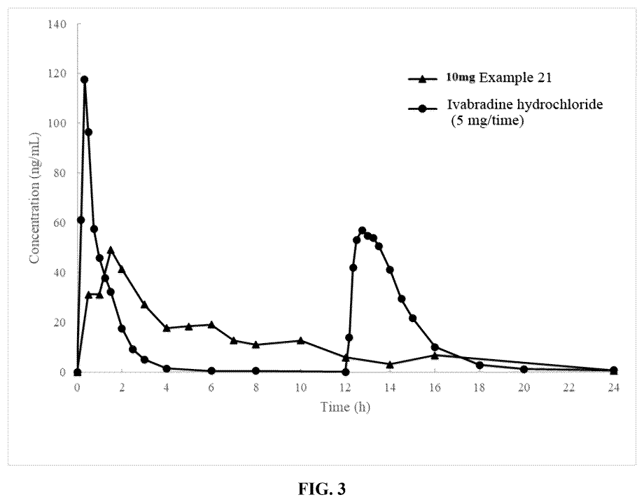

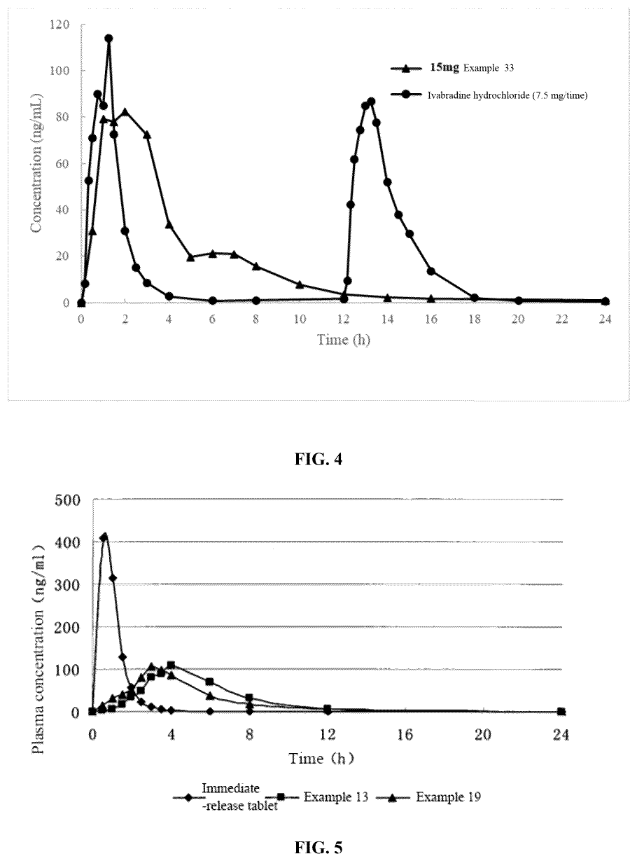

A Beagle dog pharmacokinetic comparative study was performed on samples by using the formulations in Examples 21, 30 and 33 and commercially available formulations of Ivabradine hydrochloride immediate-release tablets (produced by French company Servier, brand name: Corlentor®). Corlentor® with a specification of 5 mg was split into two half-tablets with 2.5 mg per half-tablet, and the two half-tablets were taken 12 hours apart. The two half-tablets with a specification of 2.5 mg per half-tablet were compared with 1 tablet of sample in Example 30 (containing 5 mg of Ivabradine) for comparative study. The two tablets of Corlentor® with a specification of 5 mg per tablet were taken 12 hours apart, and compared with 1 tablet of sample in Example 21 (containing 10 mg of Ivabradine) for comparative study. Corlentor® with a specification of 7.5 mg was taken 12 hours apart, and compared with 1 tablet of sample in example 33 (containing 15 mg of Ivabradine) for comparative study. The experimental results are seen in FIG. 2-FIG. 4.

According to the results in FIG. 2-FIG. 5, it can be seen that the sustained-release formulation prepared in the present disclosure achieves lower Cₘₐₓ compared with the immediate-release formulation, but is still higher than that in patent application CN102908327A, which can not only overcome the side effects that rapid Cₘₐₓ increase leads to excessive heart rate reduction after the immediate-release formulation is taken, but also ensures the effective treatment concentration. The Ivabradine hydrochloride sustained-release tablet prepared in the present disclosure reaches peak concentration about 1 h after administration, and can reach 24-hour long-acting sustained release, while the sustained-release formulation in CN102908327A reaches peak concentration about 4 h after administration and only can reach 12-hour sustained release, and therefore compared with the sustained-release formulation in CN102908327A, the Ivabradine hydrochloride sustained-release tablet prepared in the present disclosure demonstrates faster onset, longer sustained release time, and overall superior performance compared to the sustained-release formulation in CN102908327A.

It should be noted that the above embodiments are merely for illustrating the technical solution of the present disclosure but not limiting the present disclosure. Although the present disclosure is described in detail with reference to preferred embodiments, persons of ordinary skill in the art should understand that amendments or equivalent replacements made to the technical solution of the present disclosure without departing from the spirit and scope of the technical solution of the present disclosure should be included within the scope of claims of the present disclosure.

1. An Ivabradine hydrochloride sustained-release tablet, comprising:
Ivabradine hydrochloride, a gel matrix sustained-release material, a pore-forming agent and a lubricating agent;
wherein, the mass parts of raw materials are as follows:
5-15 parts of Ivabradine hydrochloride;
80-140 parts of gel matrix sustained-release material;
46-112 parts of pore-forming agent; and
4-12 parts of lubricating agent;
the gel matrix sustained-release material is hydroxypropylmethyl cellulose, wherein the hydroxypropylmethyl cellulose is selected from a mixture of two hydroxypropylmethyl celluloses with viscosity indication values of 100000 mPa·s and 5 mPa·s respectively, and a weight ratio of the hydroxypropylmethyl cellulose with a viscosity indication value of 100000 mPa·s and the hydroxypropylmethyl cellulose with a viscosity indication value of 5 mPa·s is 3:1-1:4.

2. The Ivabradine hydrochloride sustained-release tablet according to claim 1, wherein the weight ratio of the hydroxypropylmethyl cellulose with the viscosity indication value of 100000 mPa·s and the hydroxypropylmethyl cellulose with the viscosity indication value of 5 mPa·s is 1:2-1:3.

3. The Ivabradine hydrochloride sustained-release tablet according to claim 1, wherein a weight percentage of the gel matrix sustained-release material in the sustained-release tablet is 40%-70%.

4. The Ivabradine hydrochloride sustained-release tablet according to claim 3, wherein the weight percentage of the gel matrix sustained-release material in the sustained-release tablet is 50%-60%.

5. The Ivabradine hydrochloride sustained-release tablet according to claim 1, wherein the pore-forming agent is selected from one or a mixture of microscystallone and lactose.

6. The Ivabradine hydrochloride sustained-release tablet according to claim 5, wherein the pore-forming agent is a mixture of microscystallone and lactose, wherein a weight percentage of the microscystallone in the sustained-release tablet is 0%-33%, a weight percentage of the lactose in the sustained-release tablet is 0%-33%.

7. The Ivabradine hydrochloride sustained-release tablet according to claim 6, wherein the weight percentage of the microscystallone in the sustained-release tablet is 8%-25%, the weight percentage of the lactose in the sustained-release tablet is 8%-25%.

8. The Ivabradine hydrochloride sustained-release tablet according to claim 1, wherein the lubricating agent is selected from one or a mixture of colloidal silica and magnesium stearate.

9. A preparation method of the Ivabradine hydrochloride sustained-release tablet according to claim 1, comprising:
initially mixing Ivabradine hydrochloride with a pore-forming agent to form a mixture and then mixing the mixture with a gel matrix material; and then adding a lubricating agent for final mixing, and performing tableting to obtain the Ivabradine hydrochloride sustained-release tablet.
